(19) United States
(12) Patent Application Publication
(10) Pub. No.: US 2022/0003744 A1
(43) Pub. Date: Jan. 6, 2022

(54) LOW COST, BATTERYLESS AND WIRELESS PAPER MULTIPLEXING SENSOR

(71) Applicant: The Texas A&M University System, College Station, TX (US)

(72) Inventors: Jun Kameoka, College Station, TX (US); Kamran Entesari, College Station, TX (US); Onder Dincel, College Station, TX (US)

(21) Appl. No.: 17/285,739

(22) PCT Filed: Oct. 15, 2019
(86) PCT No.: PCT/US2019/056303

§ 371 Date: Apr. 15, 2021

Related U.S. Application Data
(60) Provisional application No. 62/745,681, filed on Oct. 15, 2018.

(51) Int. Cl. G01N 33/487; G01N 27/30; G01N 27/22; H01Q 1/22

(52) U.S. Cl. CPC G01N 33/48792; H01Q 1/2225; G01N 27/223; G01N 27/30

(57) ABSTRACT

This disclosure describes a multiplexing, wireless, batteiyless multiplexing paper sensor. The sensor includes a polymeric, non-cellulosic substrate that includes a radio frequency (RF) wireless communication ultra-wide band (UWB) antenna and a sensing element disposed on the polymeric, non-cellulosic substrate with and without molecular imprinted structures to immobilize specific target chemical or biological objects. The sensor is powered when a signal is received by the RF wireless communication UWB antenna and then takes measurements of a medium being monitored by the sensor. Data corresponding to the measurements is then transmitted from the sensor via the RF wireless communication UWB antenna.

### PRIORITY

This application claims the benefit of priority to U.S. Provisional Application Ser. No. 62/745,681, filed Oct. 15, 2018, which is incorporated by reference herein in its entirety.

### TECHNICAL FIELD

The present disclosure generally relates to sensors. More specifically, various embodiments relate to batteryless, substrate-supported, wireless paper based sensors.

### BACKGROUND

Sensors are typically used to monitor data in various applications, such as agricultural applications and in the health care industry. In agricultural applications, sensors may be used to monitor crop fields in order to apprise fanners of various conditions present in the crop field. In agricultural applications, large areas, such as hundreds of acres, need to be monitored. For example, soil conditions are monitored in order to ensure that the soil conditions are conducive to growing crops. Typically, the soil conditions are manually monitored, where a user moves around crop fields with a probe for insertion into the crop field. Often times, this method of manually monitoring soil conditions is time prohibitive since users need to manually probe hundreds of acres.

Moreover, in some implementations, battery-powered sensors may be used for agricultural applications. In these implementations, the sensors are placed in areas that require monitoring and gather data to determine soil conditions. However, these sensors typically are expensive, require a battery, and, if the battery does not die first, tend to decay over time due to exposure to the elements. Additionally, for large areas, such as hundreds of acres, a large number of these sensors are required. Thus, using these types of sensors are cost prohibitive. Furthermore, similar to using the probes mentioned above, no automated method exists for gathering data from these sensors. Instead, the data must be manually gathered, which creates problems similar to using probes.

Furthermore, sensors are used in the health care industry. For example, glucose test strips are used to monitor blood glucose levels. However, most commercial test strips use a glucose oxidase as recognition agents, which increases cost associated with the test strips and reduces the durability of the test strips.

### SUMMARY

Sensors made in accordance with the above have several advantages. For example, by using sensors that employ polymeric, non-cellulosic substrates, the costs associated with these types of sensors are greatly decreased. Since these types of sensors are inexpensive, hundreds of sensors may be deployed in a large area, such as a crop field, at a relatively low cost. Furthermore, since an external device, such as a drone, may be used to communicate with the sensors, a large amount of data, i.e., data from the hundreds of sensors, may be gathered in a short period of time. Moreover, since the sensors described above are made from polymeric, non-cellulosic substrates and do not require a battery, the sensors may be placed in an environment for an extended period of time without fear of degradation due to the elements or power loss due to a non-functioning battery. Moreover, in accordance with embodiments of the present disclosure, sensors that may be fabricated as described herein may be stable in air for more than eleven weeks, such as sensors that include biological or chemical sensing elements.

Embodiments of the present invention relate to a multiplexing, wireless, batteryless sensor having a printable, synthetic substrate, such as a polymeric, non-cellulosic substrate. In an embodiment, the sensor may include an ultra-wide band antenna (UWB) that may have a radio frequency (RE) receiver and a RF transmitter in communication with a multiplexing sensing element. In an embodiment of the present disclosure, the RF receiver, the RF transmitter, and the multiplexing sensing element are disposed on the printable, synthetic substrate. The UWB may be capable of operating over a broad. frequency band such as, for example, about 2.2 GHz to about 17 GHz. The multiplexing sensing element may be capable of detecting and/or monitoring different parameters, such as physical, chemical, and biological parameters. The multiplexing sensing element may be a resonator such as either an interdigitated transducer or a sandwich type capacitor. In an embodiment, during use, the multiplexing sensing element can be used to determine the amount of a physical parameter in a test sample in response to receiving a signal via the antenna.

In a further embodiment of the present disclosure, a multiplexing, wireless, battery less sensor is provided. The multiplexing, wireless, batteryless sensor may include a RF wireless communication antenna and multiple sensing elements. In an embodiment, the RF wireless communication antenna may be configured to receive an RF signal. Furthermore, in an embodiment, the sensing elements are operatively coupled to the antenna and disposed on a polymeric, non-cellulosic substrate

In another embodiment of the present disclosure, a method of monitoring a parameter is provided. In this embodiment, the parameter is monitored with a sensor that may include a polymeric, non-cellulosic substrate, a RF wireless communication antenna disposed on the polymeric, non-cellulosic substrate and a sensing element disposed opposite the RF wireless communication antenna on the polymeric, non-cellulosic substrate. In an embodiment, the method comprises positioning the sensor within a medium to be monitored such that the RF wireless communication antenna is exposed to an ambient environment and the sensing element is disposed within the medium to be monitored. In an embodiment, the sensor is activated in response to a first RF communication signal received at the RF wireless communication antenna from an external device. Furthermore, in an embodiment, the method may include monitoring the parameter within the medium where the sensing element senses an attribute shift that corresponds to the parameter in the medium. In addition, data corresponding to the sensed attribute shift may be provided to the external device via a second RF communication signal sent from the RF wireless communication antenna.

In an alternative embodiment of the present disclosure, a method of forming a molecularly imprinted sensor for sensing a molecular component having a first polymeric, non-cellulosic substrate is provided. In an embodiment, a molecular component may be combined with a monomer to form a solution and the solution is combined with an oxidant to form an electrode solution. In an embodiment, a second polymeric, non-cellulosic substrate is soaked with the electrode solution. Afterwards, a portion of the electrode solution and a portion of the second polymeric, non-cellulosic substrate may be removed from the second polymeric, non-cellulosic substrate to form an electrode, in accordance with an embodiment of the present disclosure. The electrode may then be placed onto the first polymeric, non-cellulosic substrate, in accordance with an embodiment of the present disclosure.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate possible and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 illustrates an environment in which a sensor is used to measure physical parameters of a soil environment, such as an agricultural field in which crops are grown, in accordance with an embodiment of the present disclosure.

FIGS. 2 and 3 illustrate various components and component configurations of the sensor shown with reference to FIG. 1 that facilitate the functionality of the sensor, in accordance with an embodiment of the present disclosure.

FIG. 4 illustrates a close controlled system having an air tight vessel used to investigate sensor response for a sensor disposed on a paper substrate based on moisture levels, in accordance with an embodiment of the present disclosure.

FIG. 5 illustrates humidity levels within the airtight vessel of FIG. 4 determined by the sensor disposed within the airtight vessel, in accordance with an embodiment of the present disclosure.

FIG. 6 illustrates frequency bands within which the sensor shown with reference to FIGS. 2 and 3 operates, in accordance with an embodiment of the present disclosure.

FIG. 7 shows a sensing element formed with a spray-painting technique, such as direct spray painting, in accordance with an embodiment of the present disclosure.

FIGS. 8A and 8B show a sensing element formed with a rolling printing technique, such as direct rolling printing, in accordance with an embodiment of the present disclosure.

FIG. 9 illustrates a sensing element formed with an inkjet printing technique, in accordance with an embodiment of the present disclosure.

FIG. 10 shows a correlation between moisture levels in a soil environment and an impedance measured by a sensor.

FIG. 11 shows a sensor in accordance with alternative embodiments of the present disclosure.

FIG. 12 illustrates a method of monitoring a desired parameter using a sensor in accordance with embodiments of the present disclosure.

FIG. 13 shows a method of fabricating components of a sensor that may be used to monitor biological or chemical components, in accordance with embodiments of the present disclosure.

FIG. 14 illustrates a conductive polyaniline or conductive ink electrode formed using the method described with reference to FIG. 13, in accordance with an embodiment of the present disclosure.

FIGS. 15A-15C demonstrate the correlation between components captured by the conductive polvaniline electrode shown with reference to FIG. 14 based on a measured resistance, in accordance with an embodiment of the present disclosure.

FIG. 16 shows a method for forming a conductive polyaniline electrode for a sensor that may either be a chemical or a biological sensor in accordance with embodiments of the present disclosure.

FIG. 17 illustrates the formation of conductive traces onto a substrate, where open space is left for the conductive polyaniline electrode formed in the method of FIG. 16, in accordance with an embodiment of the present disclosure.

FIG. 18 illustrates conductive polyaniline electrodes affixed to open spaces on the substrate illustrated with reference to FIG. 17, in accordance with embodiments of the present disclosure.

FIG. 19 illustrates a sensor in accordance with a further embodiment of the present disclosure.

FIGS. 20-22 illustrate frequency bands within which resonators operate in accordance with embodiments of the present disclosure.

### DETAILED DESCRIPTION

Embodiments of the present invention relate to a multiplexing, wireless, battery less sensor having a printable, synthetic substrate, such as a polymeric, non-cellulosic substrate. In an embodiment, the sensor may include an UWB that may have a RF receiver and a RF transmitter in communication with a multiplexing sensing element. In an embodiment of the present disclosure, the RF receiver, the RF transmitter, and the multiplexing sensing element are disposed on the printable, synthetic substrate. The UWB may be capable of operating over a broad frequency band such as, for example, about 2.2 GHz to about 17 GHz. The multiplexing sensing element may be capable of detecting and/or monitoring different parameters, such as physical, chemical, and biological parameters. The multiplexing sensing element may be a resonator such as either an interdigitated transducer or a sandwich type capacitor. In an embodiment, during use, the multiplexing sensing element can be used to determine the amount of a physical, chemical, and biological parameter in a test sample in response to receiving a signal via the antenna.

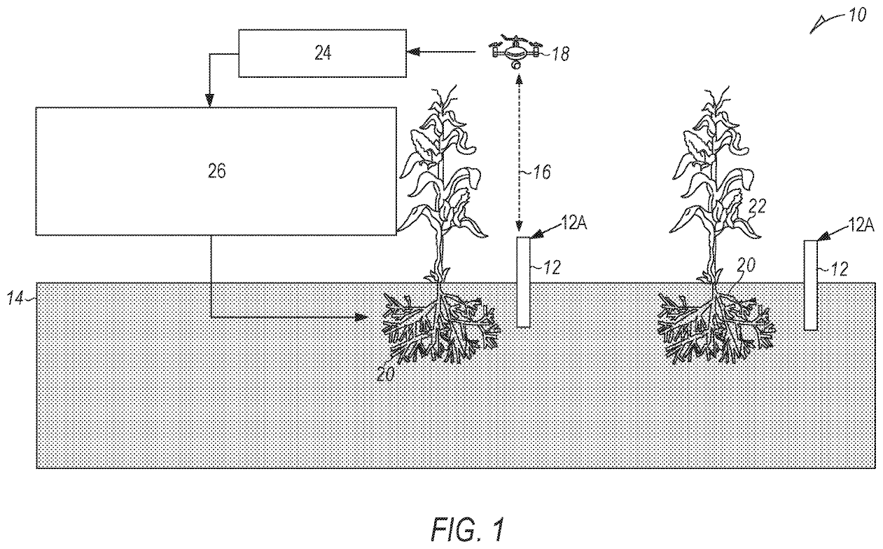

Now making reference to the Figures, and more specifically to FIG. 1, an environment 10 is shown in which a sensor 12 is used to measure physical parameters of a soil environment 14, such as an agricultural field in which crops are grown. While only two sensors 12 are shown disposed in the soil environment 14, in accordance with embodiments of the present disclosure, any number of sensors 12 may be used to monitor a given area. In accordance with an embodiment of the present disclosure, the sensor 12 multiplexes data gathered from the soil environment 14 when the sensor 12 is activated and powered on in response to RF communications 16 received from a drone 18. In particular, a portion 12A of the sensor 12 is exposed above the soil environment 14 in an ambient environment. As will be discussed further on, the sensor 12 includes componentry that facilitates the RF communications 16 between the sensor 12 and the drone 18. In this embodiment, the componentry that facilitates the RF communications 16 is located in the portion 12A of the sensor 12. Furthermore, the sensor 12 does not include any type of power source, such as a battery. Instead, the sensor 12 is powered via the RF communications 16 sent from the drone 18. An example of a drone that may be used includes a DJI Matrices 600 Pro Hexacopter drone available from DM headquartered in Shenzhen, China. In an embodiment, the drone 18 may include a high-fidelity camera system, such as a Blackmagic Design Pocket Cinema Camera 4K, available from Blackmagic Design headquartered in Melbourne, Victoria, Australia along with a gimbal system such as a Ronin-MX Gimbal Stabilizer also available from DJI. Moreover, while a drone is described as providing the RF communications 16, any type of device that communicates via RF protocols may be used to activate and power the sensor 12, in accordance with the present disclosure. For example, a smart phone, a RFID reader, any device having a Vivaldi antenna, or any other type of device capable of RF communications 16 may be used with the sensor 12. Furthermore, in embodiments where multiple sensors 12 are deployed, multiple drones 18 may be deployed to gather information from the sensors 12.

When prompted by the drone 18, the sensor 12 may monitor various physical, chemical, and biological conditions of the soil environment 14, In an embodiment, this includes the early detection of pests and diseases that may be resident in the soil environment 14. In particular, the sensor 12 may survey the physiology of roots 20 and plants 22 within the soil environment 14 to detect the presence of any pests or diseases in the soil environment 14, the roots 20, and the plants 22. In accordance with embodiments of the present disclosure, the sensor 12 may determine any type of parameter associated with the soil environment 14, the roots 20, and the plants 22. For example, the sensor 12 may determine a moisture level of the soil environment 14, a pH level of the soil environment 14, bacteria in the soil environment 14, or a temperature of the soil environment 14. Furthermore, the sensor 12 may determine nitrogen, phosphate, phosphorus, H⁺, K⁺, Ca²⁺, Mg²⁺, and/or carbon levels within the soil environment 14. It should be noted that the sensor 12 is not limited to detecting these parameters and any other types of parameters are envisioned by the present disclosure.

After the sensor 12 monitors a desired parameter in the soil environment 14, the measured parameter may be sent to the drone 18 via the RF communications 16. In an embodiment, the drone 18 may forward along the measurements to a controller 24, which may include a cloud machine learning process, artificial intelligence, or the like. The controller 24 processes the measured parameter to determine, in a scenario where the measured parameter is indicative of an undesirable condition in the soil environment 14, what type of corrective action should be taken to remediate the soil environment 14 and/or the roots 20 and the plants 22. In some embodiments, the controller 24 may employ artificial intelligence to make this determination. To further illustrate, if the sensor 12 returns a measurement indicative of a low moisture content in the soil environment 14, the controller 24 may adjust an irrigation system (indicated at 26) associated with the environment 10 in order to increase the moisture content of the soil environment 14 to a desired level. As a further example, if the sensor 12 determines that one of a nitrogen, phosphate, and/or carbon levels within the soil environment 14 are not at a proper level, the controller 24 may adjust the amount of nutrients (indicated at 26) provided to the soil environment 14. In another example, if the sensor 12 determines the onset of pests or disease, the controller 24 may adjust the amount of pesticide or any other form of remediation (indicated at 26) provided to the soil environment 14 in order to address the onset of the pests or disease. While the sensor 12 is described as being used with the soil environment 14 in an agricultural field, deployment of the sensor 12 is not limited to agricultural field applications. More specifically, in accordance with embodiments of the present disclosure, the sensor may be a biological sensor used for livestock management, such as for the early detection of viruses or bacterial infections. In addition, the sensor may be used for environmental monitoring, weather monitoring, infant monitoring, or any other type of monitoring. Moreover, the sensor may be a chemical sensor where the sensor may be an ion sensor or a molecular sensor. Also, in accordance with further embodiments of the present disclosure, a strain sensor can be made using the techniques described herein in order to monitor constricted structures such as a building, bridges, a tunnel, or any other type of structure.

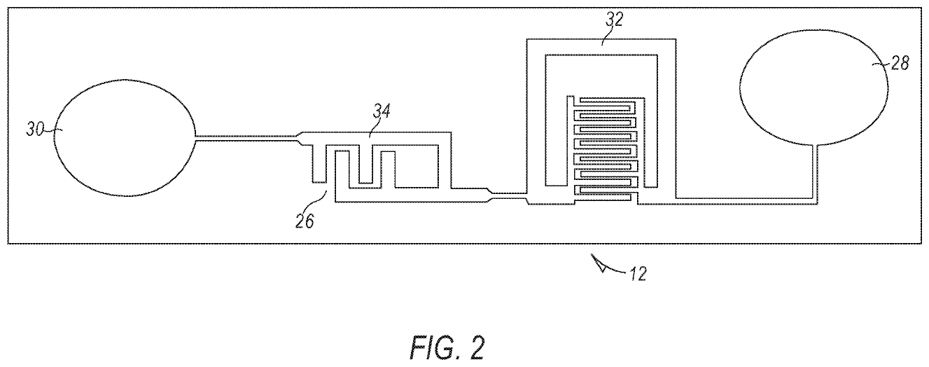

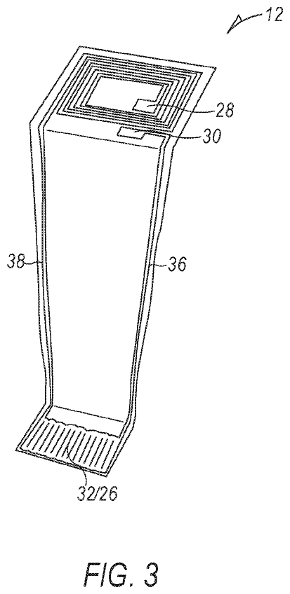

The sensor 12 includes various componenuy in order to monitor a desired parameter of the soil environment 14 and communicate with the drone 18, as shown with reference to FIGS. 2 and 3. Making reference to FIG. 2, the sensor 12 may include a substrate 26 with a receiver 28, a transceiver 30, and resonators 32 and 34 disposed on the substrate 26. In accordance with embodiments of the present disclosure, the substrate 26 may be a polymeric, non-cellulosic substrate, such as paper. In addition, the substrate 26 may be formed from an elastomeric. Examples of materials that may be used for the substrate 26 include polyethylene terephthalate (PET), polyester, polypropylene, polyvinylidene fluoride, or any other type of synthetic polymer that is resistive to bacterial digestion in the soil environment 14 in comparison to cellulose based paper. In an embodiment of the present disclosure, the substrate 26 may have a dielectric constant of 3.26 with a loss tangent of 0.1.

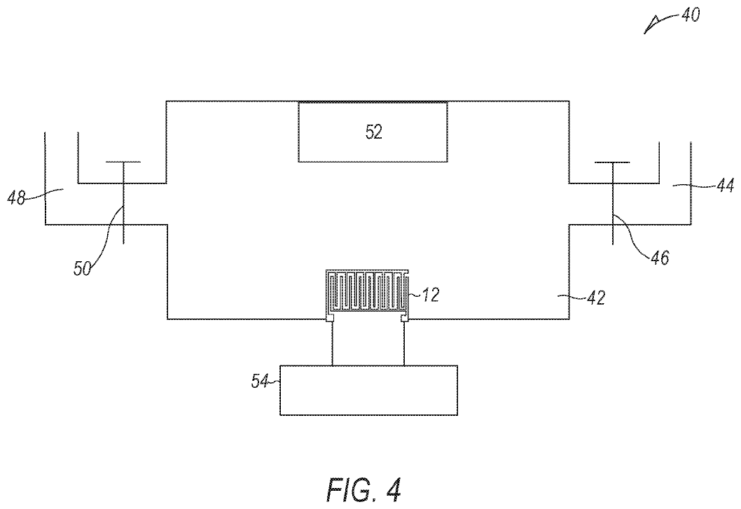

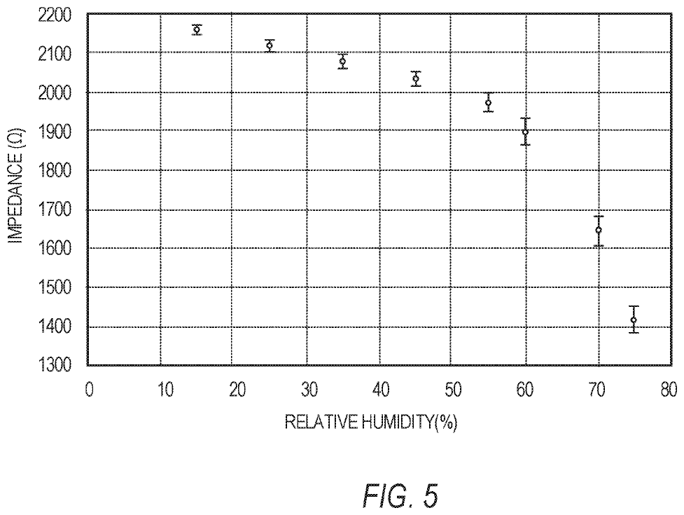

Simulations with PET substrates indicate little performance loss in comparison to traditional materials used for device substrates, such as PCB. In particular, FIG. 4 illustrates a closed controlled system 40 used to investigate sensor response for a sensor having a paper substrate, such as the sensor 12 that includes the substrate 26, based on moisture levels. Here, nitrogen is released into an airtight vessel 42 of the closed control system 40 as shown at 44 via an airtight vessel inlet 46 to decrease moisture levels in the airtight vessel 42. Cold mist 48 is also released into the airtight vessel 42 via an airtight vessel inlet 50 in order to increase a relative humidity level inside the airtight vessel 42. A commercially available moisture sensor 52 is placed in the airtight vessel 42 to monitor moisture levels within the airtight vessel 42 and monitor the capabilities of the sensor 12 within the environment created in the airtight vessel 42 at low frequencies. Furthermore, a network analyzer 54 defines a frequency response of the sensor 12 at different moisture levels within the airtight vessel 42. As shown with reference to FIG. 5, as humidity levels within the airtight vessel 42 increase, the sensor 12, which includes the substrate 26 which is a polymeric, non-cellulosic substrate, accurately determines the increased humidity levels based on the impedance values determined at the sensor 12.

Returning to FIGS. 2 and 3, the receiver 28 and the transceiver 30 together form a RF wireless communication UWB for the sensor 12. Furthermore, in some embodiments, the receiver 28 and the transceiver 30 may be formed in the portion 12A of the sensor 12 as discussed above. The receiver 28 may be any device capable of receiving RF signals from an external source, such as the drone 18, a smart phone, a RFID reader, or any other type of device capable of sending and receiving RF signals. The transceiver 30 may be any device capable of transmitting RF signals to an external device, such as the drone 18, a smart phone, a RFID reader, or any other type of device capable of sending and receiving RF signals. As mentioned above, the sensor 12 does not include any type of power source, such as a battery. Instead, in some embodiments, the sensor operates as a passive RFID tag, where, when the sensor 12 receives radio waves via the RF communications 16, the antenna forms a magnetic field and the sensor 12 draws power from the magnetic field. As such, the structure of the sensor 12 is simplified and costs associated with manufacturing the sensor 12 are greatly reduced.

Each of the resonators 32 and 34 form individual sensing elements that may be configured to sense various parameters, such as physical, chemical, and biological parameters. The resonators 32 and 34 are multiplexing resonators which, as will be discussed further below, monitor a desired parameter based on this multiplexing capability. While the resonators 32 and 34 are shown as interdigitated transducers, any type of device capable of functioning as a resonator may be used such as sandwich type capacitive sensors that sense capacitance changes. Additionally, in accordance with embodiments of the present disclosure, the resonators may be formed by electrodes and molecularly imprinted structures such that the sensing element includes two sensing elements, an electrode and a molecularly imprinted structure. In these embodiments, the molecularly imprinted structure may be integrated into the electrode, above the electrode, or below the electrode. Moreover, devices that may be used for the resonators may include any type of LC circuit, inclusive of a tank circuit or tuned circuit, or any type of device that employs an inductor and a capacitor. Furthermore, the resonators may include either capacitive or conductive parallel plates. Moreover, in accordance with further embodiments of the present disclosure, in embodiments where conductive ink is used to form the resonators, the conductive ink may be modulated with a molecular imprinting process in order to immobilize specific targets, such as molecules, cells, viruses, or the like.

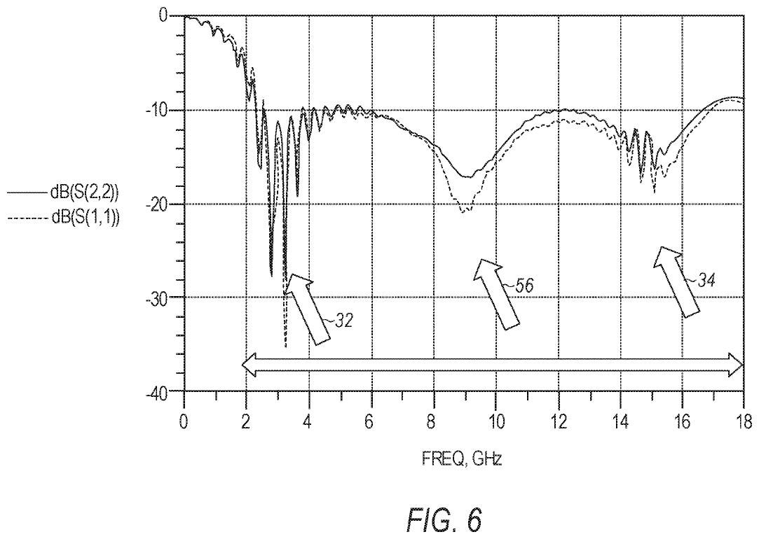

While the sensor 12 is shown having two resonators, the resonators 32 and 34, any number of resonators may be used, in accordance with some embodiments. Moreover, the type of resonators used for the sensor 12 may be selected based on a required operating frequency for the sensor 12. For example, the sensor 12 may operate over a broad frequency band such as, for example, 2.2 GHz to 17 GHz. Resonators may be selected for the sensor 12 that facilitate operation over this communication band. To further illustrate, making reference to FIG. 6, a frequency band between 2.0 GHz and 18 GHz for a sensor, such as the sensor 12 is shown. Here, arrows 32 and 34 correspond to the resonators 32 and 34, respectively, while the arrow 56 corresponds to a third resonator. As may be seen with reference to the Figure, the resonator 32 provides frequency band operation at the lower end of the frequency band, i.e., from 2.0 GHz to 6 GHz, while the resonator 34 provides frequency band operation at the higher end of the frequency band, i.e., from 14 GHz to 18 GHz. Further, the resonator corresponding to the arrow 56 provides frequency operation between the lower and higher ends of the frequency bands, i.e., between 6 GHz and 14 GHz. As noted above, the sensor 12 may include multiple resonators. In an embodiment, different resonators operating at different frequency bands, such as the resonators 32 and 34 along with a resonator denoted at the arrow 56, may be provided on a single sensor, such as the sensor 12. Thus, a single sensor may operate over a broad frequency band and may communicate with different devices, i.e., different drones 18, etc., that operate at different frequencies. In other words, the sensor 12 may be capable of communicating with multiple communication devices via the RF communications 16.

Returning to FIGS. 2 and 3, the antenna formed by the receiver 28 and the transceiver 30 couples with resonators, such as the resonators 32 and 34, via trace lines 36 and 38, as shown with reference to FIG. 3, In accordance with embodiments of the present disclosure, the trace lines 36 and 38 may be formed from conductive polymers as well as conductive metal paint such as silver, gold, or copper, on synthetic papers as either of these will function in the soil environment 14 for an extended period of time. In other embodiments, the trace lines 36 and 38 may be a wire, or may be formed from carbon particles.

The sensor 12 may be formed using any number of methods. These methods may include spray painting, rolling printing, and inkjet printing, as shown with reference to FIGS. 7-9. FIG. 7 shows a sensing element, such as one of the resonators 32 and 34, formed in accordance with a spray painting technique, such as direct spray painting. Here, a 3D printed mask structure may be formed and then placed over a substrate, such as the substrate 26. Conductive materials, i.e., conductive ink, such as silver coated copper, silver particles, gold particles, copper particles, or the like, may be sprayed on the substrate where the 3D printed mask structure may be used as a shadow mask. In accordance with an embodiment of the present disclosure, the conductive ink may have a conductivity in a range of 0.35×10⁶ Siemens/m to about 0.35×10⁶ Siemens/m. Examples of conductive ink that may be used include NovaCentrix JS ADEV 291 ink-jet ink available from Novacentrix, which is headquartered in Austin, Texas. It should be noted that in embodiments where conductive ink is used as described above, the trace lines 36 and 38 may be fowled with conductive ink. The 3D printed mask structure prevents the leakage of conductive ink onto other structures, such as other electrode structures.

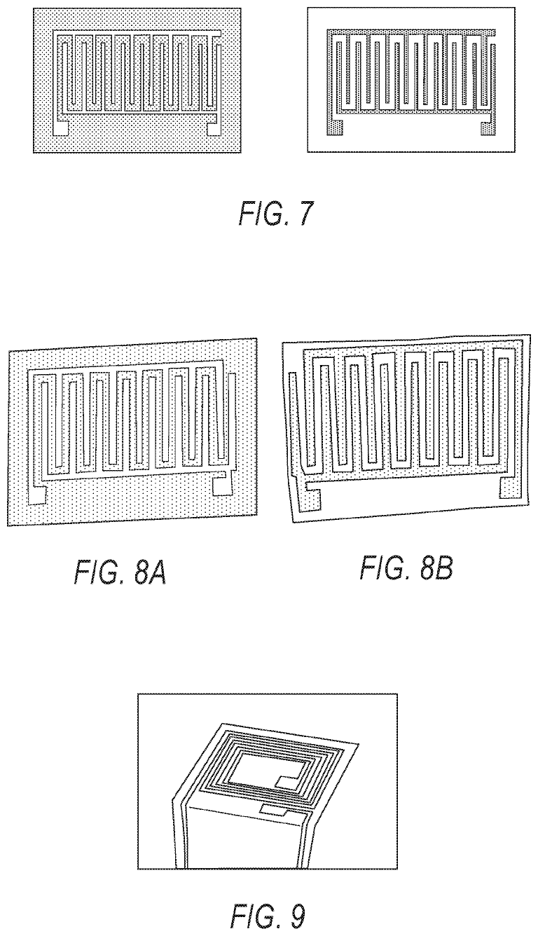

In addition to spray painting, a rolling printing technique may be used to form the sensor 12. FIGS. 8A and 89 show a sensing element, such as one of the resonators 32 and 34, formed in accordance with a rolling printing technique, such as direct rolling printing. In this embodiment, a mask, such as a paper mask or a tape mask, may be patterned on a substrate, such as the substrate 26, with a CO₂ laser cutter. After formation of the mask on the substrate, a roller, which is immersed in a paint bath that may include a conductive material as described above, is brushed over the mask on the substrate, to form a sensor as shown with regards to 8A and SIB.

Furthermore, inkjet printing, such as direct inkjet printing, may be used to form the sensor, as shown in FIG. 9. In this embodiment, an inkjet cartridge is tilled with solvent that has slightly diluted conductive paint having silver micro-nano particles. The substrate 26, which in this embodiment may be polyester paper, is used as the paper source for the inkjet printer. An electrode design is then printed with the inkjet printer, as shown with reference to FIG. 9. Thus, in some embodiments, the fabrication process for the sensing elements of the sensor 12 is based on the direct inkjet-printing method. Here, conductive metal paint or a conductive polymer is printed directly by an ink-jet printer as electrodes, i.e., the resonators 32, 34, on the substrate 26, after hydrophilic conversion process of substrate 26.

Moreover, in embodiments where moisture levels or pH levels are being measured by the sensor 12, prior to printing the elements of the sensor 12, such as the resonator 32 and/or the resonator 34, the substrate 26 is soaked in moisture and pH sensitive materials such as hydrogel or silica gel prior to subjecting the substrate 26 to the printing processes discussed above.

As noted above, the sensor 12 is activated and powered up when the sensor 12 receives the RE communications 16 from the drone 18. In particular, the receiver 28 receives the RF communications 26 and the sensor 12 is activated. Upon activation, the sensor 12 monitors and then determines a parameter in the soil environment 14 by sensing an attribute shift, such as an impedance shift, a frequency shift, and/or a time domain shift. As an example, in accordance with an embodiment of the present disclosure, where a moisture level of the soil environment 14 is being monitored, a pulse signal received via the receiver 28 is injected into a resonator of the sensor 12, such as one of the resonators 32 and 34. As noted above, in accordance with an embodiment of the present disclosure, the resonator, such as one of the resonators 32 and 34, is a multiplexing resonator. In an embodiment, the resonator of the sensor 12, such as one of the resonators 32 and 34, may feed the pulse signal to both a reference line of the resonator and a capacitance line of the resonator. The moisture level in the soil environment may cause the pulse signal to shift in the capacitance line of the resonator relative to the pulse in the reference line. In accordance with an embodiment, the phase shift correlates to a moisture level in the soil environment 14. In particular, in accordance with an embodiment of the present disclosure, the phase shift may be used to determine an impedance. This data may be transmitted as a sensor signal to the transceiver 30, which transmits the sensor signal to the drone 18. A non-linear load impedance of the transceiver 30 (including the capacitive load of one of the resonators 32 and 34) reflects back the second harmonic of the received signal modulated by the sensor signal to the drone 18 to prevent interference between backscattered and transmitted RF signals in the RF communications 16. The drone 18 extracts the sensing information, i.e., information relating to impedance, from the back-scattered RF signal.

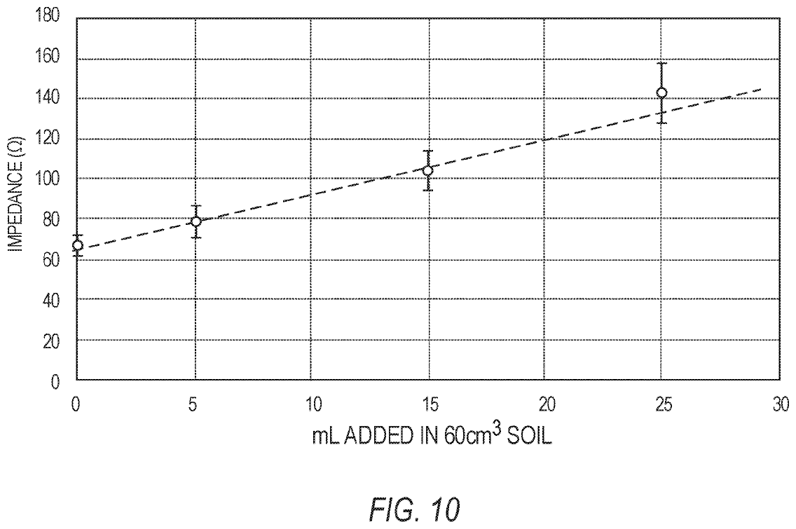

In some embodiments, the impedance determined based on the phase shifts correlates to a moisture level within the soil environment 14, as shown with reference to FIG. 10. For example, as may be seen with reference to FIG. 10, as the moisture level of the soil environment 14 increases, an impedance measured by the sensor 12 increases. Thus, in this example, if the moisture levels are determined as excessively high, the controller 24 may control the irrigation system to decrease the amount of irrigation provided to the soil environment 14, the roots 20, and the plants 22 for a given period of time. Conversely, if the moisture levels are determined as being too low, the controller 24 may control the irrigation system to increase the amount of irrigation provided to the soil environment 14, the roots 20, and the plants 22 for a given period of time.

Using similar principles, a relative humidity or a pH level of the soil environment 14 may also be determined. As noted above, in embodiments where the sensor 12 is used to measure humidity or pH levels, the substrate 26 is soaked in hydrogel or silica gel. Soil moisture and pH directly influences the dielectric constant and thickness of hydrogel materials, and therefore, moisture and pH in soil can be detected by sensing an impedance shift, a frequency shift, and/or a time domain shift of the resonator 32 and/or the resonator 34, as discussed above. Moreover, using similar principles, the sensor 12 may be mechanical sensors, such as a strain sensor where the sensing element is deformed by an external force and the deformation causes an impedance change.

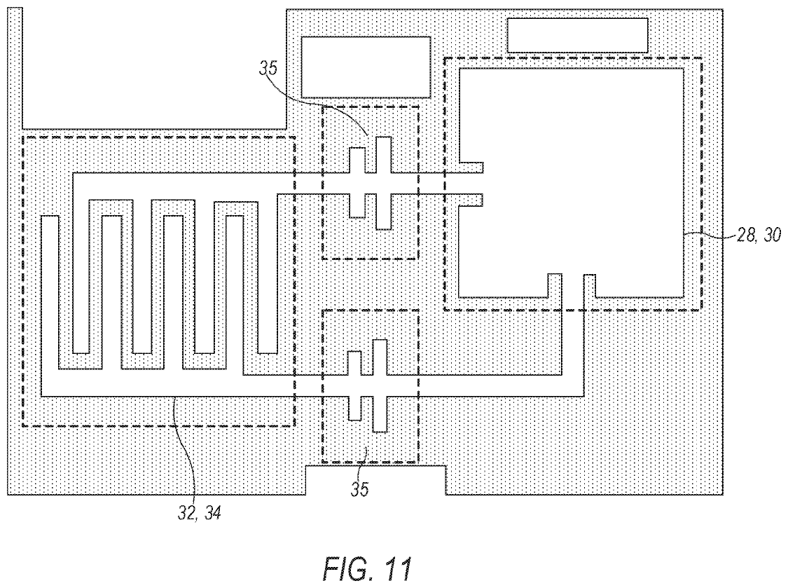

It should be noted that while resonators are described as the sensing elements for the sensor 12, sensing elements for the sensor 12 may be implemented in other ways. For example, electro chemical ion-selective sensors may be printed on the substrate 26. Furthermore, in accordance with alternative embodiments of the present disclosure, the sensor 12 may include matching networks 35 disposed between the antenna formed by the receiver 28 and the transceiver 30 and the resonator 32 and/or the resonator 34, as shown with reference to FIG. 11. In accordance with an embodiment of the present disclosure, the matching networks 35 function to minimize signal loss between the antenna formed by the receiver 28 and the transceiver 30 and the resonator 32 and/or the resonator 34. In accordance with embodiments of the present disclosure, the matching networks may be any component that minimizes signal loss along a signal pathway.

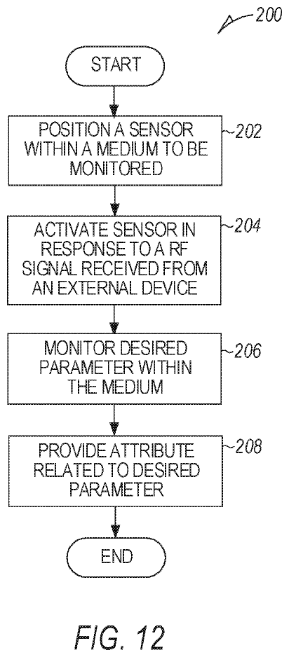

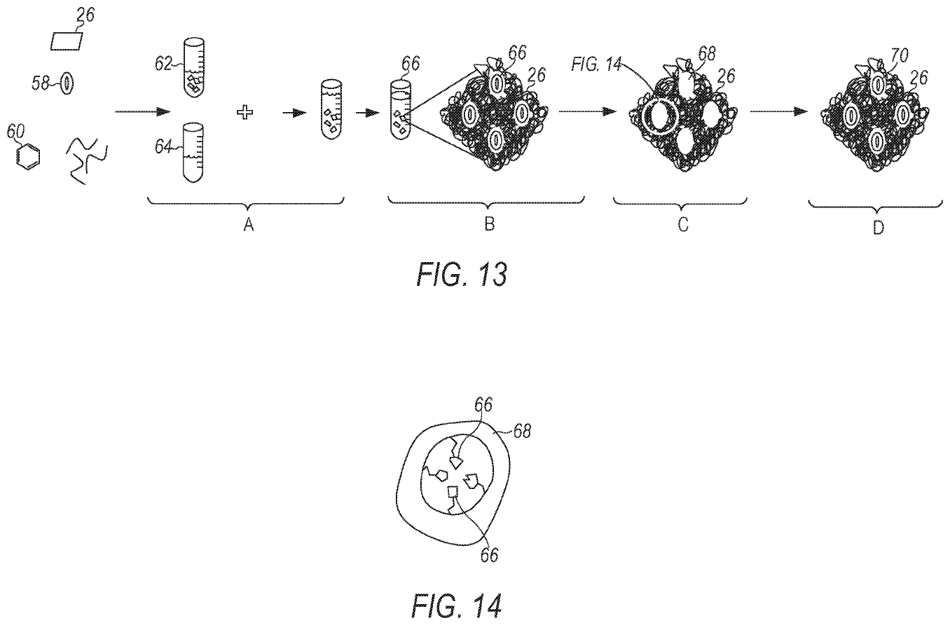

Now making reference to FIG. 12, a method 200 for monitoring a desired parameter using the sensor 12 in accordance with embodiments of the present disclosure is discussed. Initially, in an operation 202, a sensor is positioned within a medium to be monitored. For example, a sensor may be placed in an agricultural field to measure aspects of the soil of the agricultural field, in a livestock enclosure, or in an outside environment to measure weather, the environment, or the like. As an example, making reference to FIG. 1, in the operation 202, the sensor 12 is placed in the soil environment 14, where the top portion 12A of the sensor 12 is above-ground and exposed in an ambient environment in order to facilitate RF communications. Moreover, the sensing elements of the sensor 12, i.e., the resonator 32 and/or the resonator 34, are placed in the soil environment 14 in order measure moisture levels of the soil environment 14.

After the sensor is positioned in the operation 202, the sensor is activated in response to a RF communication signal received from an external device in an operation 204. After activation, the sensor commences monitoring of a desired parameter within the medium in an operation 206. In the example, after the sensor 12 is placed in the soil environment, the sensor 12 may be activated via the RF communications 16 received from an external device, such as the drone 18, in the operation 204. At this point, the sensor 12 may monitor a desired parameter in the soil environment in the operation 206 as discussed above. In this example, the sensor 12 is to be used to determine a moisture level of the soil environment 14. Thus, upon activation by the drone 18, the sensor 12 measures a moisture level of the soil environment 14, as detailed above.

After the sensor monitors the desired parameter in the operation 206, the sensor provides an attribute that relates to measurement data related to the desired parameter in an operation 208. Returning to the example, in the operation 206, the sensor 12 determines a moisture level of the soil environment 14. As discussed above, the sensor 12 may determine the moisture level by sensing an attribute shift, such as an impedance shift, a frequency shift, and/or a time domain shift when a pulse signal from the RF communications 16 is provided to the resonator 32 and/or the resonator 34. This attribute, which may be used to determine the moisture level of the soil environment 14, is sent to the drone 18 via another RE communication signal in the operation 208. The drone 18 then forwards the data to the controller 24 in order to determine if remediation is required, as detailed above. After the sensor 12 sends the data to the drone 18, the method 200 is complete.

In addition to measuring physical parameters, such as moisture and humidity, the sensor 12 may also be used to measure chemical and biological components in the soil environment 14. Moreover, the sensor 12 may be used to measure chemical and biological components for other environments, such as humans and atmospheric air quality. More specifically, molecular imprinting (MIP) technology is used to create a binding site for biological or chemical material, such as glucose, on a conductive polyaniline (PANI) electrode. In this embodiment, the biological or chemical material, such as glucose, is used to bind components in an environment being tested with the PANI electrode, which includes the biological or chemical material. The binding of the biological material on a MIP surface changes the electrical properties of the PANI electrode and the sample by changing a charge carrier density.

Now making reference to FIG. 13, a method of fabricating an electrode that may be used to monitor biological or chemical components is shown, in accordance with embodiments of the present disclosure. In this embodiment, the sensor 12 is being formed to test glucose levels, where the substrate 26 is imprinted with a test sample 58 and a monomer 60. In this example, the test sample 58 may be glucose and the monomer 60 may be aniline. However, in accordance with embodiments of the present invention, the test sample 58 may be any biological or chemical component, such as any type of bacteria or virus, nitrogen, phosphate, carbon, or the like. Furthermore, the test sample 58 may include other biological organisms such as biomolecules, and biomarkers such as protein. In addition, the monomer 60 may be any type of precursor, such as hydrogel, a polymer, a conductive ink, or the like. As shown at operation (A) in FIG. 13, the test sample 58 and the monomer 60 are combined to a form a test sample/monomer solution 62. Afterwards, the substrate 26 is dipped into the test sample/monomer solution 62. At an operation (B), the test sample/monomer solution 62 is combined with an oxidant 64 to form a PAM solution 66. In accordance with an embodiment of the present disclosure, the substrate remains in the test sample/monomer solution 62 as the test sample/monomer solution 62 is combined with the oxidant 64 to form the PAM solution 66. In an alternative embodiment, the PANI solution is first formed and then the substrate is dipped into the PANI solution.

In an operation (C), portions of the testing component 66 are removed to form PANI electrodes 68 within the substrate 26. After removal of the portions of the testing components 66, portions of the testing components 66 may remain within the PANI electrodes 68 such that testing components are molecularly imprinted within the PANI electrodes 68, as more clearly shown with reference to FIG. 14.

When a sensor such as the sensor 12 shown with reference to FIGS. 13 and 14 is used to monitor a desired parameter, the sensor 12 captures components being monitored by the sensor. For example, at an operation (D), the sensor 12 captures components 70. The sensor 12 is activated and powered up when the sensor 12 receives the RF communications 16. As noted above, in addition to the drone 18, handheld devices, such as a smart phone, a RFID reader, or any other type of device capable of RF communications 16 may be used with the sensor 12. Upon activation via a drone, a smart phone, a RFID reader, or any other type of device capable of RF communications 16, the sensor 12 monitors and then determines an amount of components within the PANI electrode 68. In accordance with these embodiments, resistance levels measured. by the PANI electrodes 68 change based on glucose concentrations in a test sample. For example, when the sensor 12 captures the components 70, these components, which may correspond to molecules, change the resistance of the PANI electrode 68. To further illustrate, when a pulse signal is received at the receiver 28 and is injected into the PANI electrode 68, the components 70 will either increase or decrease resistance, based on the type of component and the amount. In accordance with another embodiment of the present disclosure, the sensor 12 may include a further electrode, which may be used to calculate the resistance based on the components 70.

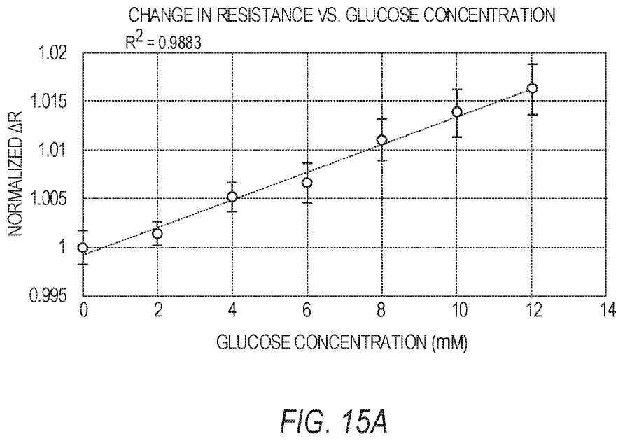

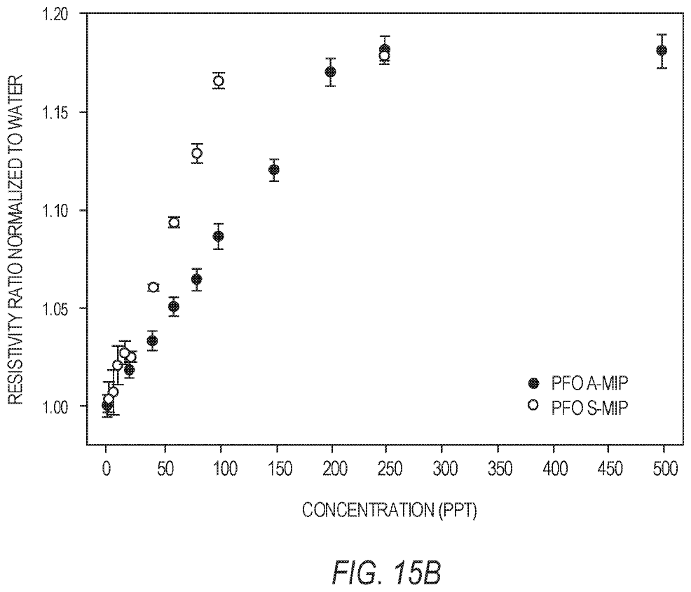

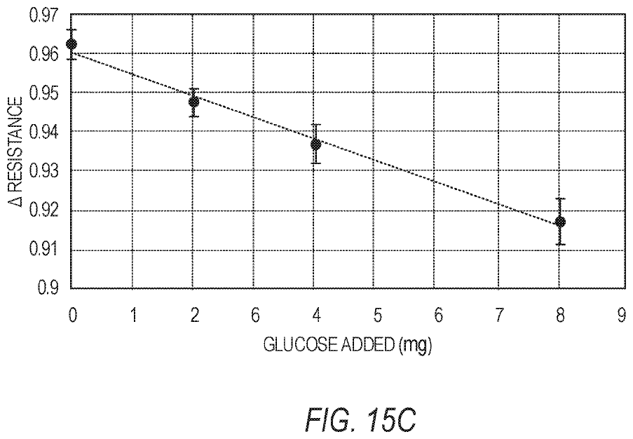

In either embodiment, as may be seen with reference to FIG. 15A, in accordance with embodiments of the present disclosure, the higher the concentration of the components 70 within the PANI electrode 68, such as glucose, the higher resistance. Additionally, when other testing components, such as perfluorooctanoic acid (PFO-A) or perfluorooctanesulfonic acid (PFO-S) are used, resistance increases as concentration levels of PFO-A or PFO-S increase, as shown with reference to FIG. 15B. Furthermore, in some implementations, such as in instances where bovine glucose is being measured, resistance decreases as glucose levels increase, as illustrated with reference to FIG. 15C. The calculated resistance values may be used to determine a concentration value of the components 70 in the sample being tested. While glucose is described with reference to FIGS. 13 and 14, any type of chemical or biological components may be tested with the sensor 12 as described above. For example, in an embodiment where airborne viruses or bacteria are being tested, instead of glucose, the virus or bacteria may be implanted in the substrate 26 as discussed above with regards to FIGS. 13 and 14. In these embodiments, the sensor 12 may be used externally, for example in a room, to determine whether or not a virus or bacteria exists in the tested space. To further illustrate, as mentioned above, the sensor 12 may be used for livestock management to facilitate, among other implementations, the early detection of viruses or bacterial infections. Sensors implemented in accordance with FIGS. 13 and 14 may be used for these livestock scenarios.

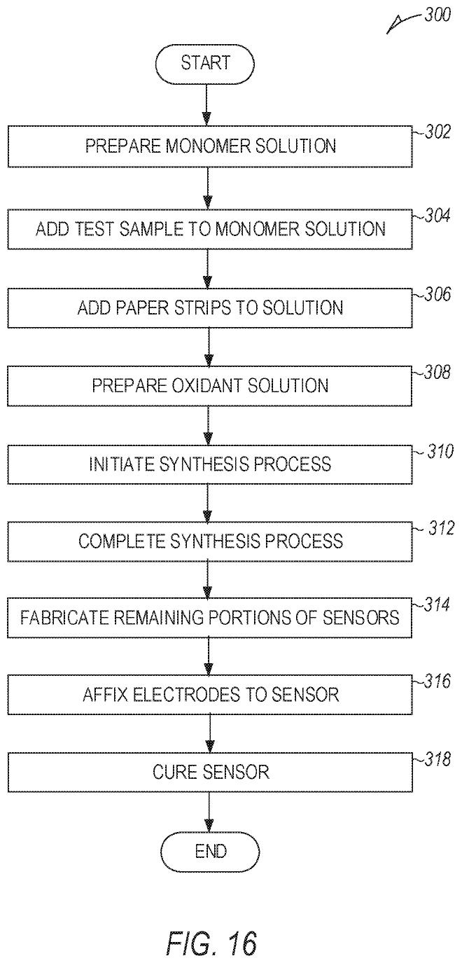

Now making reference to FIG. 16, a method for forming an electrode for a sensor that may either be a chemical or a biological sensor is illustrated in accordance with embodiments of the present disclosure. Initially, ire an operation 302, a monomer solution is prepared. For example, a PANI electrode is synthesized by preparing a monomer solution where, in an embodiment, 250 uL of an aniline solution is blended with 1 mL of hydrochloric acid (HCl). In this embodiment, HCl may function as a doping agent. In an embodiment, the use of HCl as a doping agent increases the conductivity of a PANI electrode formed according to FIG. 13. In this example, the aniline/HCl solution is added to a 3 mL solution of distilled (DI) water to adjust a concentration of the aniline/HCl solution.

After the monomer solution is formed in the operation 302, a test sample is added to the monomer solution in an operation 304. Returning to the example, in an embodiment where the test sample 58 is glucose, a template molecule, which may be 50 mg of glucose, is blended with the aniline/HCl solution to create glucose binding sites. In an embodiment, in order to achieve a proper concentration for synthesis, DI water is added until the total volume of the solution becomes 5 ml.

Once the test sample is added to the monomer solution in the operation 304, paper strips are added to the monomer solution in an operation 306. In the example, in an embodiment, paper strips, such as paper strips having the characteristics of the substrate 26 detailed above, are dipped into the monomer solution in order to be soaked with the monomer solution. In an embodiment, the paper strips are dipped in the monomer solution in order to saturate the paper strip. In an embodiment, the paper strips may remain in the solution for at least 10 minutes before the oxidation process begins and may be stirred to ensure continuous saturation.

An operation 308 is then performed where an oxidant solution is prepared. Returning to the example, in accordance with an embodiment, in order to prepare the oxidant solution, 0.609 mL of HCl is added to a 4 mL vial of DI water. Furthermore, 409 mg of ammonium persulfate is added to the oxidant solution. After 10 seconds of stirring, additional DI water is added in order to create a solution volume of 5 mL. In some embodiments, HCl may be used to maintain pH levels and doping levels during the synthesis process.

After the oxidant solution is prepared in the operation 308, a synthesis operation is initiated in an operation 310 and then completed in an operation 312. In the example, once the oxidant solution is prepared, a polyaniline synthesis process is initiated. In the example, the monomer solution is stirred and the oxidant solution is added drop by drop to the monomer solution. In some embodiments, the polyaniline synthesis process may be sensitive to the rate at which the oxidant solution is added. Therefore, in an embodiment, drops of oxidant may be dispensed in 5 second intervals from a pipette until the color of solution changes to a dark blue. In an embodiment, during the synthesis operation, the paper strip remains immersed in the solution. During the synthesis operation, PANI is formed on the paper strip. In order to complete the synthesis process in the operation 312, the paper strips are removed from the solution and washed with DI water to remove excess PANI. During this operation, portions of the PANI structure are removed from the paper strips to form the PANI electrodes 68. Furthermore, in this embodiment, the paper strips are left out to dry for at least 8 hours before usage. At this point, the electrode is complete and ready for integration with the sensor.

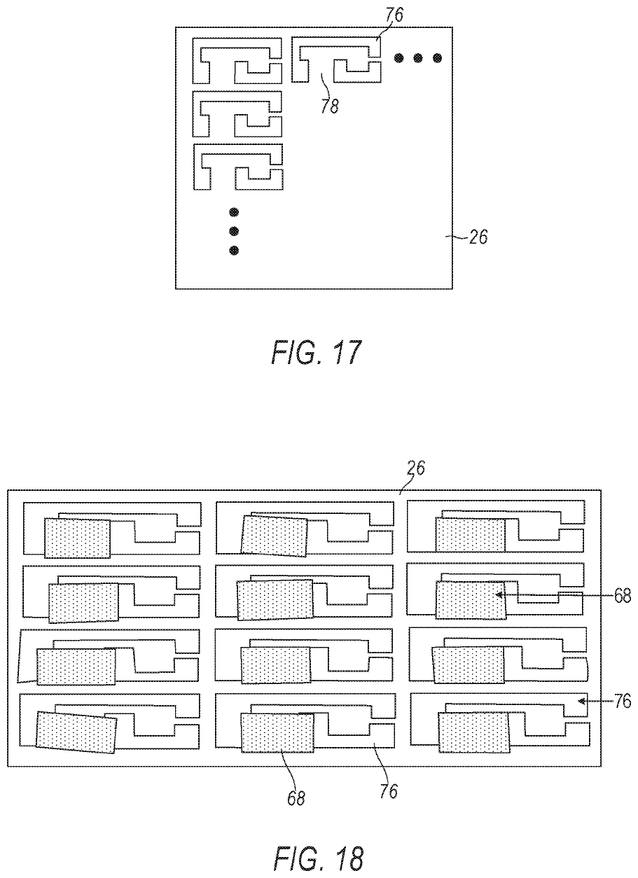

After the synthesis process is complete and the PANI electrode is fabricated in the operation 312, the remaining portion of the sensor 12 is fabricated in an operation 314. In the example, the sensor 12 may be formed using a printing operation, such as inkjet printing, where the sensor 12, minus the PANI electrode, is printed onto the substrate 26 with a Fujifilm Dimatix Material Printer 2830 available from Fujifilm, which is headquartered in Minato, Tokyo, Japan in accordance with the settings set forth below with regards to Table 1. In an embodiment, the inkjet printer may use Novacentrix JS ADEV 291 ink available from NovaCentrix, which is headquartered in Austin, Tex. For example, making reference to FIG. 17, conductive traces 76, such as silver traces, are printed onto the substrate 26, where open spaces 78 are left for the PANI electrodes 68 formed in the operations 310 and 312, in accordance with an embodiment of the present disclosure.

TABLE 1
Summary of the settings used in the material
printer for silver electrode printing.

| | Parameter | | | | |
|---|---|---|---|---|---|
| | Nozzle | Nozzles | Nozzle | | Cartridge |
| | Spacing | Activated | Diameter | Frequency | Size |
| Value | 254 μm | 16 | 21 μm | 30 kHz | 10 pL |

| | Parameter | | | | |
|---|---|---|---|---|---|
| | Substrate | Jetting | Applied | Drop | Drop |
| | Temperature | Frequency | Voltage | Spacing | Angle |
| Value | 30° C. | 5 Hz | 30 V | 20 μm | 4.4° |

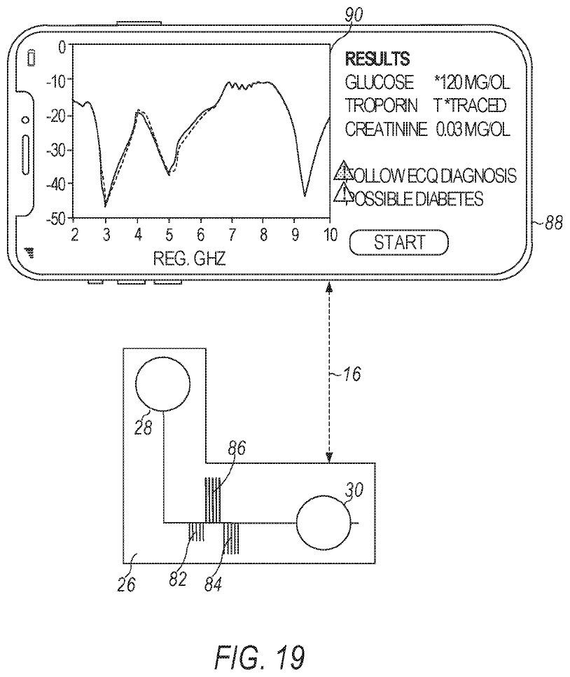

After completion of the remaining portions of the sensor 12 in the operation 314, the PANI electrodes are affixed to the sensor 12 in an operation 316. In the example, making reference to FIG. 18, in an embodiment, the PANI electrodes 68 are affixed at the open spaces 78 on the substrate 26. In an embodiment, in order to improve connection between the conductive traces 76 and the PANI electrodes 68, silver paste is deposited on the connected area using a fine-tipped brush.

When the PANI electrodes 68 are affixed to the sensor 12, the sensors 12 are cured in order to solidify the connections in an operation 318. For example, in accordance with an embodiment of the present disclosure, the devices may be cured in an oven at 140° C. for one hour.

Now making reference to FIG. 19, this Figure shows the sensor 12 in accordance with an alternative embodiment of the present disclosure. Here, the sensor 12 may include three resonators, resonators 82, 84, and 86. The resonators 82, 84, and 86 operate in a manner similar to the resonators discussed above. Furthermore, each of the resonators 82, 84, and 86 may be formed using the techniques described above with reference to FIGS. 13-18. In other words, the resonators 82, 84, and 86 may be used to monitor biological and/or chemical components. For example, the resonator 82 may be used to monitor glucose levels, the resonator 84 may be used to monitor troponin levels, and the resonator 86 may be used to monitor creatinine levels. It should be noted that the resonators 82, 84, and 86 are not limited to monitoring these components. In particular, the resonators 82, 84, and 86 may be used to monitor any number of biological and/or chemical components.

In an embodiment, the sensor 12 may be activated in response to the RF communications 16 received from a device 88. In accordance with embodiments of the present disclosure, the device 88 may be a smart phone. However, the device 88 may be any device capable of RF communications, such as a RFID reader, a drone, or any other type of device capable of RF communications 16 with the sensor 12. Once activated, the sensor 12 proceeds to monitor all of the biological and/or chemical components the resonators 82, 84, and 86 are capable of monitoring for, such as glucose, troponin, and creatinine. Afterwards, the results may be sent via the RF communications 16 to the device 88, which shows the results on a display 90. In some embodiments, the display 90 may he a graphical user interface.

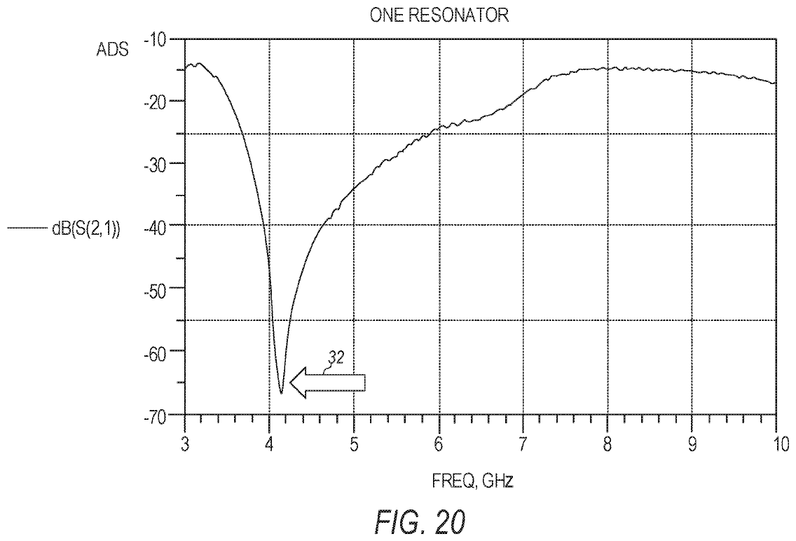

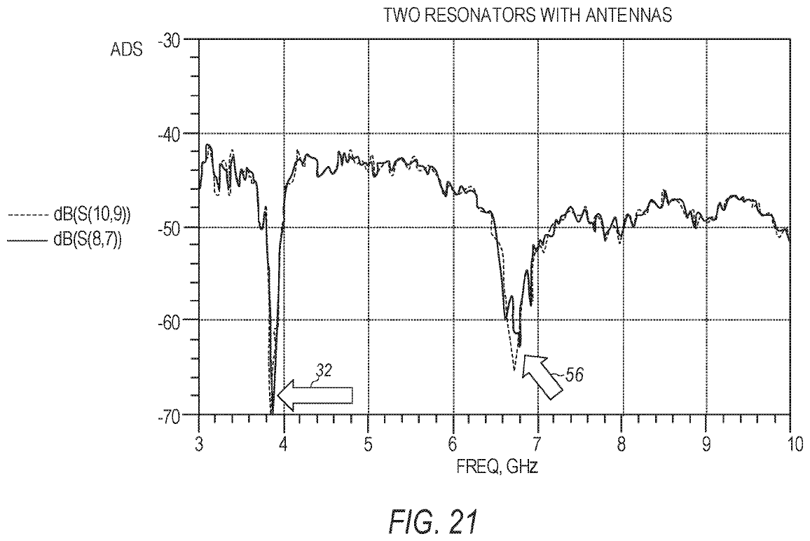

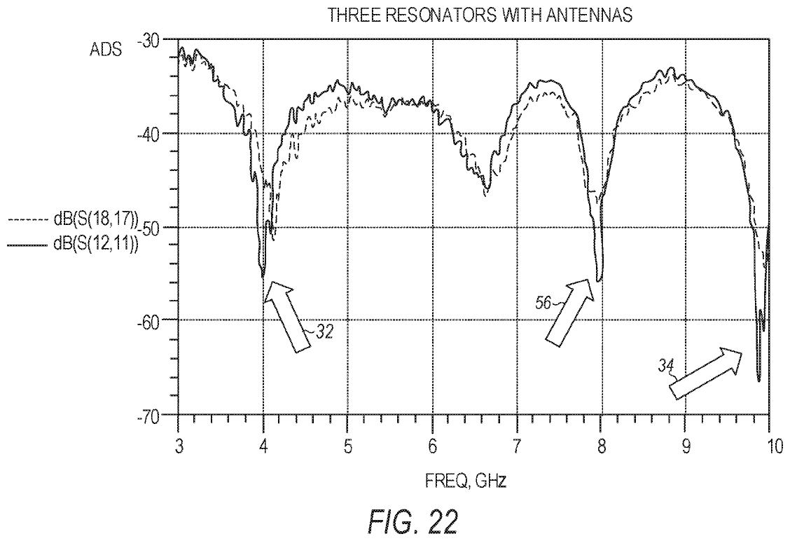

Now making reference to FIGS. 20-22, the frequency bands at which resonators, such as the resonators 32 and 34 and a resonator that corresponds to arrow 56, operate in conjunction with an antenna, are shown in accordance with embodiments of the present disclosure. Making reference to FIG. 20, the operating frequency of the resonator 32 is shown. In particular, when a sensor includes the resonator 32, the resonator 32 may operate at the lower end of the frequency band, i.e., from 2.0 GHz to 6 GHz during operation of the antenna for the sensor 12 described above. With regard to FIG. 21, shown is an embodiment where the sensor includes two resonators, shown with arrows 32 and 56. Here, in accordance with an embodiment, the resonator 32 operates between about 3.5 GHz and about 4.5 GHz while a resonator corresponding to arrow 56 operates between about 6.5 GHz and about 7.5 GHz during operation of the antenna for the sensor 12 described above. FIG. 22 illustrates an embodiment where a sensor includes three resonators, shown with arrows 32, 34, and 56. In this embodiment, the resonator 32 operates between about 3.5 GHz and about 4.0 GHz while a resonator corresponding to arrow 56 operates between about 6.0 GHz and about 8.0 GHz during operation of the antenna for the sensor 12 described above. Moreover, in the embodiment shown with reference to FIG. 22, a resonator corresponding to arrow 34 operates between about 9.5 GHz and about 10 GHz during operation of the antenna for the sensor 12 described above. In accordance with the embodiments described herein, sensors in accordance with embodiments of the present disclosure are able to operate in a range between about 3 GHz and about 8 GHz in addition to the ranges described herein.

Having described various aspects and features of the inventive subject matter, the following numbered examples are provided as illustrative embodiments:

1. A multiplexing, wireless, battery-less sensor comprising a radio frequency (RF) wireless communication ultra wideband (UWB) antenna configured to receive an RF signal; and a sensing element operatively coupled to the antenna and disposed on a polymeric, non-cellulosic substrate.

2. The sensor of example 1, wherein the RF signal is in a range of 2.2 GHz to 17 GHz.

3. The sensor of example 1, wherein the polymeric, non-cellulosic substrate comprises polyester, polyethylene, polypropylene, or polyvinylidene fluoride.

4. The sensor of example 1, wherein the sensing element comprises at least one of a moisture sensor, a mechanical sensor, a biological sensor, and a chemical sensor.

5. The sensor of example 4, wherein the biological sensor is a bacteria sensor,

6. The sensor of example 4, wherein the chemical sensor is an ion or molecular sensor.

7. The sensor of any one of examples 1-6, wherein the at least one of a moisture sensor, a biological, a mechanical sensor, and a chemical sensor are parallel plates (capacitive or conductive) or interdigitated.

8. The sensor of any one of examples 1-6, wherein the sensing element is operatively coupled to the antenna via a conductive polymer, a conductive ink film, or a wire.

9. The sensor of example 1, wherein the antenna is located on a first portion of the polymeric substrate and the sensing element is spaced from the UWB antenna and located at a second portion of the polymeric substrate.

10. The sensor of example 1, wherein the sensing element is a sensor printed on the polymeric substrate.

11. The sensor of example 1, wherein the sensing element comprises a conductive polymer, a conductive ink, or metal paint.

12. The sensor of example 11, wherein the metal paint comprises silver, gold, carbon or copper particles.

13. The sensor of example 1, wherein the sensing element is a capacitor.

14. The sensor of example 1, wherein the sensing element comprises a resonator.

15. The sensor of example 14, wherein the sensor further comprises a matching network associated with the resonator and the RF wireless communication antenna.

16. The sensor of example 14, wherein the sensing element comprises an additional resonator.

17. The sensor of example 1, wherein the UWB wireless communication antenna includes a receiver and a transceiver.

18. The sensor of example 1, wherein the sensor further comprises an additional sensing element, where each of the sensing elements monitors for separate components.

19. The sensor of example 1, wherein the sensing element is a multiplexing sensing element and includes a first sensing element formed by an electrode and a second sensing element formed by a molecularly imprinted structure.

20. A method for detecting changes in at least one of moisture levels, concentration of biological organisms, and levels of chemicals with the sensor of example 1, the method comprising receiving an RF signal by the antenna, wherein the RF signal is transmitted to the sensor, receiving a sensor signal in response to the received RF signal, reflecting a second harmonic of the received signal, modulated by the sensor signal, and extracting sensing information from a back-scattered RF signal.

21. The method of example 20, wherein the RF signal is transmitted to the sensor via a drone, a RF transmitter, or a smart phone.

22. The method of example 20 or example 21, wherein the RF signal is in a range of 2.2 GHz to 17 GHz.

23. The method of any one of examples 20-22, wherein the chemicals comprise ions or molecules.

24. The method of example 23, wherein the molecules or ions comprise nitrogen, H⁺, K⁺, Ca²⁺, Mg²⁺ or combinations thereof.

25. The method of any one of examples 20-22 wherein the biological organisms comprise bacteria, biomolecules, or biomarkers such as protein.

26. A method of making the sensor of example 1, the method comprising depositing a conductive ink on a substrate, curing the conductive ink; and connecting the antenna to the cured conductive ink.

Although embodiments have been described with reference to specific example embodiments, it will be evident that various modifications and changes may be made to these embodiments without departing from the broader scope of the invention. Accordingly, the specification and drawings are to be regarded in an illustrative rather than a restrictive sense. The accompanying drawings that form a part hereof show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This description, therefore, is not to be taken in a limiting sense, and the scope of various embodiments is defined only by the appended claims, along with the full range of equivalents to which such claims are entitled.

1. A multiplexing, wireless, batteryless sensor comprising:
a radio frequency (RF) wireless communication ultra wideband (UWB) antenna. configured to receive an RF signal; and
at least one sensing element operatively coupled to the antenna and disposed on a polymeric, non-cellulosic substrate.

2. (canceled)

3. The sensor of claim 1, wherein the polymeric, non-cellulosic substrate comprises polyester, polyethylene, polypropylene, or polyvinylidene fluoride.

4. The sensor of claim 1, wherein the sensing element comprises at least one of a moisture sensor, a mechanical sensor, a biological sensor, and a chemical sensor,

5. (canceled)

6. (canceled)

7. (canceled)

8. The sensor of claim 1, wherein the sensing element is a sensor printed on the polymeric substrate and is operatively coupled to the antenna via a conductive polymer, a conductive ink paint, a metal paint or a wire.

9. The sensor of claim 1, wherein the antenna is located on a first portion of the polymeric substrate and the sensing element is spaced from the antenna and located at a second portion of the polymeric substrate.

10. (canceled)

11. (canceled)

12. (canceled)

13. The sensor of claim 1, wherein the sensing element is a capacitor or resistor.

14. The sensor of claim 1, wherein the sensing element comprises a resonator formed from one of a conductive polymer, a conductive ink or a metal paint.

15. (canceled)

16. (canceled)

17. (canceled)

18. (canceled)

19. The sensor of claim 1, wherein the sensing element is a multiplexing sensing element and includes a first sensing element formed by an electrode and a second sensing element formed by a molecularly imprinted structure.

20. A method for detecting changes in at least one of moisture levels, concentration of biological organisms, and levels of chemicals with the sensor of claim 1, the method comprising:
receiving an RF signal by the antenna, wherein the RF signal is transmitted to the sensor;
receiving a sensor signal in response to the received RF signal;
reflecting a second harmonic of the received signal, modulated by the sensor signal; and
extracting sensing information from a back-scattered RF signal.

21. (canceled)

22. (canceled)

23. (canceled)

24. (canceled)

25. (canceled)

26. (canceled)

27. A method of monitoring a parameter using a sensor having a polymeric, non-cellulosic substrate, a radio frequency (RF) wireless communication antenna disposed on the polymeric, non-cellulosic substrate and a sensing element disposed opposite the RF wireless communication antenna on the polymeric, non-cellulosic substrate, the method comprising:
positioning the sensor within a medium to be monitored such that the RF wireless communication antenna is exposed to an ambient environment and the sensing element is disposed within the medium to be monitored;
activating the sensor in response to a first RF communication signal received at the RF wireless communication signal antenna from an external device;
monitoring the parameter within the medium where the sensing element senses an attribute shift that corresponds to the parameter in the medium; and
providing data corresponding to the sensed attribute shift to the external device via a second RF communication signal sent from the RF wireless communication antenna.

28. (canceled)

29. The method of claim 27, wherein the polymeric, non-cellulosic substrate comprises polyester, polyethylene, polypropylene, or polyvinylidene fluoride.

30. The method of claim 27, wherein the sensing element comprises at least one of a moisture sensor, a mechanical sensor, a biological sensor, and a chemical sensor.

31. The method of claim 27, wherein the sensing element is operatively coupled to the antenna via a conductive polymer, a conductive ink, a metal paint or a wire.

32. The method of claim 27, wherein the sensing element comprises a conductive polymer or metal paint.

33. (canceled)

34. (canceled)

35. The method of claim 27, wherein the sensing element includes an electrode and molecularly imprinted structure.

36. (canceled)

37. (canceled)

38. (canceled)

39. A method of forming a molecularly imprinted sensor for sensing a molecular component, the sensor having a first polymeric, non-cellulosic substrate, the method comprising:
combining the molecular component with a monomer to form a solution;
combining the solution with an oxidant to form an electrode solution;
providing the electrode solution onto a second polymeric, non-cellulosic substrate;
removing a portion of the electrode solution and a portion of the second polymeric, non-cellulosic substrate from the second polymeric, non-cellulosic substrate to form an electrode; and
placing the electrode onto the first polymeric, non-cellulosic substrate.

40. The method of claim 39, wherein the molecular component is selected from at least one of a chemical component and biological component.

41. (canceled)

42. (canceled)

43. The method of claim 39, wherein the monomer is aniline or conductive ink.

44. The method of claim 39, wherein the operation of providing the electrode solution onto the second polymeric, non-cellulosic substrate further comprises dipping the second polymeric, non-cellulosic substrate into the solution and combining the solution with the oxidant to form the electrode solution when the second polymeric, non-cellulosic substrate is in the solution.

45. The method of claim 39, wherein the sensor further comprises a sensing element formed by the electrode and is molecularly imprinted.

46. (canceled)

47. (canceled)
